(19) United States
(12) Patent Application Publication
(10) Pub. No.: US 2010/0150975 A1
(43) Pub. Date: Jun. 17, 2010

(54) Structured Composition Comprising an Encapsulated Active

(76) Inventors: Jiten Odhavji Dihora, Hamilton, OH (US); Shaffiq Amin Jaffer, Loveland, OH (US); Anna Vadimovna Radomyselski, Loveland, OH (US); Phillip Kyle Vinson, Fairfield, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/581,941

(22) Filed: Oct. 20, 2009

Related U.S. Application Data
(60) Provisional application No. 61/106,723, filed on Oct. 20, 2008.

(51) Int. Cl. A61K 8/02; A61K 9/48; A61K 9/50; A01N 25/28; A61L 9/01; A61Q 17/04; A61P 17/16; A01P 1/00; A01P 17/00; A61K 8/11; C11D 3/22; A61Q 13/00; A61Q 5/10

(52) U.S. Cl. 424/401; 424/451; 424/490; 424/408; 424/76.1; 424/59; 512/4; 510/405; 8/404

(57) ABSTRACT

A structured liquid composition having a liquid matrix having water and a fibrous network structurant, the fibrous network structurant having a non-polymeric crystalline hydroxyl-functional materials; a bacterial cellulose network; and mixtures thereof; and an encapsulated active. The encapsulated active is suspended within the liquid matrix of the structured liquid composition such that the encapsulated actives do not undesirably sink, float, or collect in any discrete portion of the composition. Further, the structured liquid composition can be in the form of a raw material such as an encapsulated active slurry, in the form of an additive such as a laundry additive, and as a consumer end product. In one embodiment, the encapsulated active is a perfume microcapsule.

### CROSS REFERENCE TO RELATED APPLICATION

This application claims the benefit of priority to U.S. Provisional Application Ser. No. 61/106,723, filed Oct. 20, 2008 which is herein incorporated by reference.

### BACKGROUND OF THE INVENTION

The encapsulation of functional actives, also known as benefit agents, is known using micro and or nano capsules. These encapsulated actives are suitable for a variety of applications, including laundry products, hard surface cleaning compositions, air freshening products, beauty care compositions, and so forth. These encapsulated actives are often made into an aqueous slurry form during manufacturing and can even be commercialized and consumed by end users as said encapsulated active slurry. These encapsulated active slurries can be used in various capacities depending on the amount of encapsulated active desired; for example, as a raw material for incorporation into other end product compositions, can be used as additives such as laundry additives, or can be used as an end product for consumers such as in an air freshening or flavoring capacity.

One problem encountered with the production of these the encapsulated active slurry is that the distribution of the encapsulated actives within the liquid matrix needs to be controlled so that the encapsulated actives do not overly float, sink or otherwise gravitate towards discreet sections of the liquid matrix during processing, when packaged for later processing with other ingredients, or when in a packaged consumer product. In order to properly disperse and suspend the encapsulated actives with the liquid matrix, structuring agents can be introduced into the composition. There are number of compositions which can provide structuring benefits is large. Structuring agents can be separated into internal structuring agents, and external structuring agents. Where the internal structuring agents provide functional benefits to the composition in addition to the structuring capability, and where external structurants are not included to provide any functional benefits to the composition other than structuring.

The problem with known external structuring agents is that they are sensitive to pH levels as well as electrolyte levels (i.e. salts). Certain types of encapsulated active slurries have “high” levels of salts resulting in compositional compatibility problems with many known structuring agents. Known external structuring agents include polymers or gums structurants, many of which are known to swell or expand when hydrated to form random dispersion of independent microgel particles. Examples of polymers and gums structurants include: gellan gum, pectine, alginate, arabinogalactan, caageenan, xanthum gum, guar gum, rhamsan gum, furcellaran gum, carboxymethylcellulose and cellulose. See e.g. U.S. Pat. No. 6,258,771 to Hsu et al. U.S. Pat. No. 6,077,816 to Puvvada et al. U.S. Patent Publ. No. 2005/0203213 to Pommiers et al.; and WO 2006/116099 to Fleckenstein et al. Although gums have been used to provide structuring benefits, the gums have been found to be undesirable due to any pH and electrolyte level sensitivity which can decrease their structuring capacity or lead to other undesirable issues such as composition opacity or gelling or clumping of product.

Another composition reported to provide structuring benefits is cellulose, i.e. bacterial celluloses. Conventional uses of bacterial celluloses include improving rheological properties for hydraulic fracturing fluids used for hydraulic fracturing of geological formations; addition to well bore drilling muds; and as a food ingredient. See e.g. U.S. Pat. Nos. 5,350,528, 5,362,713, and 5,366,750. The bacterial cellulose is typically cultured using a bacterial strain of Acetobacter aceti var. xylinum and dried using spray drying or freeze drying techniques. Attempts to manufacture and prepare the dried bacterial cellulose compositions which can be rehydrated and activated into a bacterial cellulose network for use in end products are known. Examples of these attempts are provided in U.S. Pat. No. 6,967,027 to Heux et al. and U.S. Patent Publ. Nos. 2007/0027108: 2008/0108714 and 2007/197779. Difficulties around the use of these types of external structurants are that they often need very high energy intensity processing in order to turn the dried composition into an activated form in aqueous solution.

Despite the many types of compositions which can be used as external structurants for various types of compositions, there has not been reported a suitable external structurant for incorporation into an encapsulated active slurry as described herein.

### SUMMARY OF THE INVENTION

One aspect of the present invention provides for a structured liquid composition comprising: a liquid matrix comprising: a fibrous network structurant at a level of from about 0.05% to about 1% by weight of said composition, said fibrous network structurant comprising: a non-polymeric crystalline hydroxyl-functional materials; a bacterial cellulose network; and mixtures thereof; and water at a level of from about 0.1 to about 75% by weight of said composition; and a plurality of encapsulated actives at a level of from about 0.5% to about 70% by weight of said composition.

Another aspect of the present invention provides for a process of making a structured liquid composition comprising an encapsulated active comprising: supplying a fibrous network structurant; supplying a encapsulated active slurry; and combining said fibrous network structurant with said encapsulated active slurry to form a mixture; subjecting said mixture to a pressure drop below 100 psi across a mixing chamber to form said composition.

Yet another aspect of the present invention provides for a method of making a structured composition comprising: allowing a user to select a type of composition; supplying a fibrous network structurant; supplying an encapsulated active slurry based on the selection made in step a); and combining said fibrous network structurant with said encapsulated active slurry to form a structured composition.

### DETAILED DESCRIPTION OF THE INVENTION

It has importantly and surprisingly been found that the use of cellulosic structurants, capable of forming reticulated fibrous networks do not suffer from the compositional incompatibility when incorporated into the encapsulated active slurry. Further, it has been found that this class of external structurant is less susceptible to composition incompatibility compared to earlier attempts to structure said compositions. Furthermore, by structuring the encapsulated active slurry, there is less need to include additional mixing processing which can fracture or otherwise undesirable damage the composition. Moreover, it has importantly been found that where the cellulosic structurant is combined with an aqueous composition in a premix process, the cellulosic structurant can form a fibrous network structurant to form a liquid matrix without undesirably subjecting said encapsulated actives to excessive processing forces which can damage the capsules. This embodiment comprising a premixing step, then includes a step where the encapsulated actives are combined with the liquid matrix and mixed in a less intense processing environment.

### DEFINITIONS

As used herein “consumer product” means baby care, beauty care, fabric & home care, family care, feminine care, health care, snack and/or beverage products or devices intended to be used or consumed in the form in which it is sold, and not intended for subsequent commercial manufacture or modification. Such products include but are not limited to diapers, bibs, wipes; products for and/or methods relating to treating hair (human, dog, and/or cat), including, bleaching, coloring, dyeing, conditioning, shampooing, styling; deodorants and antiperspirants; personal cleansing; cosmetics; skin care including application of creams, lotions, and other topically applied products for consumer use; and shaving products, products for and/or methods relating to treating fabrics, hard surfaces and any other surfaces in the area of fabric and home care, including: air care, car care, dishwashing, fabric conditioning (including softening), laundry detergency, laundry and rinse additive and/or care, hard surface cleaning and/or treatment, and other cleaning for consumer or institutional use; products and/or methods relating to bath tissue, facial tissue, paper handkerchiefs, and/or paper towels; tampons, feminine napkins; products and/or methods relating to oral care including toothpastes, tooth gels, tooth rinses, denture adhesives, tooth whitening; over-the-counter health care including cough and cold remedies, pain relievers, RX pharmaceuticals, pet health and nutrition, and water purification; processed food products intended primarily for consumption between customary meals or as a meal accompaniment (non-limiting examples include potato chips, tortilla chips, popcorn, pretzels, corn chips, cereal bars, vegetable chips or crisps, snack mixes, party mixes, multigrain chips, snack crackers, cheese snacks, pork rinds, corn snacks, pellet snacks, extruded snacks and bagel chips); and coffee.

As used herein, the term “cleaning composition” includes, unless otherwise indicated, granular or powder-form all-purpose or “heavy-duty” washing agents, especially cleaning detergents; liquid, gel or paste-form all-purpose washing agents, especially the so-called heavy-duty liquid types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, cleaning bars, mouthwashes, denture cleaners, dentifrice, car or carpet shampoos, bathroom cleaners; hair shampoos and hair-rinses; shower gels and foam baths and metal cleaners; as well as cleaning auxiliaries such as bleach additives and “stain-stick” or pre-treat types, substrate-laden products such as dryer added sheets, dry and wetted wipes and pads, nonwoven substrates, and sponges; as well as sprays and mists.

As used herein, the term “fabric care composition” includes, unless otherwise indicated, fabric softening compositions, fabric enhancing compositions, fabric freshening compositions and combinations there of.

As used herein, the phrase “encapsulated active” encompasses a benefit agent or active agent core material and a wall material that at least partially surrounds the benefit agent or active agent core material; encompasses microcapsules with a benefit agent or active agent core material; encompasses microcapsules including perfume microcapsules; encompasses matrix materials such as a benefit agent surrounded at least partially by a solid or gelled carrier; encompasses matrix materials such as a benefit agent at least partially surrounded by a wall or wall-like network; encompasses aggregates of two materials where one material at least partially surrounds the other. Further, “active” includes both “benefit agent” and “active agents.”

As used herein, the term “particle” is synonymous with the phrase “encapsulated active”.

As used herein, the articles “a” and “an” when used in a claim, are understood to mean one or more of what is claimed or described.

As used herein, the terms “include”, “includes” and “including” are meant to be non-limiting.

As used herein, “intense high shear processing conditions” means a mixing step sufficient to activate the bacterial cellulose and provide the requisite yield stress of the present invention.

As used herein, “liquid matrix” refers to the liquid components of the present liquid detergent composition, where measurements made on the liquid matrix are performed in the absence of any suspension particles.

As used herein, a “structurant” is any material which is added to the composition to provide rheological and structuring benefits, for example as measured by yield stress. As used herein, “external structurant” means a material which has as its primary function that of providing rheological alteration to the liquid matrix. Generally, therefore, an external structurant will not, in and of itself, provide any significant cleaning benefits or any significant ingredient solubilization benefits. An external structurant is thus distinct from an internal structurant which may also alter matrix rheology but which has been incorporated into the liquid composition for some additional or alternative primary purpose.

As used herein, all tests and measurements, unless otherwise specified, are made at 25° C.

The test methods disclosed in the Test Methods Section of the present application should be used to determine the respective values of the parameters of Applicants' inventions.

Unless otherwise noted, all component or composition levels are in reference to the active portion of that component or composition, and are exclusive of impurities, for example, residual solvents or by-products, which may be present in commercially available sources of such components or compositions.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All documents cited are, in relevant part, incorporated herein by reference; the citation of any document is not to be construed as an admission that it is prior art with respect to the present invention.

1. Liquid Matrix Comprising an External Structuring System

The composition of the present invention comprises a liquid matrix comprising from about 0.05% to about 6.0% of an external structuring system, alternatively less than about 0.125%, alternatively less than about 0.05%, alternatively less than about 0.01% of said external structuring system, alternatively at least about 0.01%, alternatively at least about 0.05%, by weight of the composition. The external structuring system for use in with the present invention comprises a fibrous network structurant and optionally an additional structurant such as a polymer and/or gum structurant. The external structurant is a fibrous network structurant and is at a level of from about 0.05% to about 1% by weight of said composition, alternatively from about 0.1% to about 0.5%, alternatively from about 0.2% to about 0.3%. The fibrous network structurant comprises a hydrophobically modified polymer, such as hydrogenated castor oil; a bacterial cellulose network, such as a microfibrous bacterial cellulose, and mixtures thereof.

It has importantly been found that the use the fibrous network structurant allows for structuring of encapsulated active compositions while avoiding one or more of the problems encountered with the addition of other external structurants. Without intending to be bound by theory, it is believed that other external structurants such as the polymers and/or gum structurants can be sensitive to the pH levels and salt concentrations present in many encapsulated active slurries. It has been found that the fibrous network structurant provides the rheological benefits desired, such as shear thinning and encapsulated active suspension without undesirable side-affects from the pH and salt levels.

The bacterial cellulose network, also known as microfibrillated bacterial cellulose, comprises individual bacterial cellulose fibers which are activated in the presence of water to form a reticulated network of fibers which are believed to be hydrogen bonded. In one embodiment, the external structuring system consists essentially of a bacterial cellulose network.

a. Bacterial Cellulose Network

The external structuring system of the present invention comprises a bacterial cellulose network at a level of up to about 100%, alternatively up to about 99%, alternatively up to about 95%, alternatively up to about 80%, alternatively up to about 70% by weight of said external structuring system. The term “bacterial cellulose” is intended to encompass any type of cellulose produced via fermentation of a bacteria of the genus Acetobacter and includes materials referred popularly as microfibrillated cellulose, reticulated bacterial cellulose, and the like.

The bacterial cellulose network is formed by processing of a mixture of the bacterial cellulose in a hydrophilic solvent, such as water, polyols (e.g., ethylene glycol, glycerin, polyethylene glycol, etc.), or mixtures thereof. This processing is called “activation” and comprises, generally, high pressure homogenization and/or high shear mixing. It has importantly been found that activating the bacterial cellulose under sufficiently intense processing conditions provides for increased yield stress at given levels of bacterial cellulose network. Yield stress, as defined below, is a measure of the force required to initiate flow in a gel-like system. It is believed that yield stress is indicative of the suspension ability of the liquid composition, as well as the ability to remain in situ after application to a vertical surface.

Activation is a process in which the 3-dimensional structure of the bacterial cellulose is modified such that the cellulose imparts functionality to the base solvent or solvent mixture in which the activation occurs, or to a composition to which the activated cellulose is added. Functionality includes providing such properties as shear-thickening, imparting yield stress-suspension properties, freeze-thaw and heat stability, and the like. The processing that is followed during the activation process does significantly more than to just disperse the cellulose in base solvent. Such intense processing “teases apart” the cellulose fibers to expand the cellulose fibers. The activation of the bacterial cellulose expands the cellulose portion to create a bacterial cellulose network, which is a reticulated network of highly intermeshed fibers with a very high surface area. The activated reticulated bacterial cellulose possesses an extremely high surface area that is thought to be at least 200-fold higher than conventional microcrystalline cellulose (i.e., cellulose provided by plant sources).

The bacterial cellulose utilized herein may be of any type associated with the fermentation product of Acetobacter genus microorganisms, and was previously available, one example, from CPKelco U.S. is CELLULON®. Such aerobic cultured products are characterized by a highly reticulated, branching interconnected network of fibers that are insoluble in water. The preparation of such bacterial cellulose products are well known and typically involve a method for producing reticulated bacterial cellulose aerobically, under agitated culture conditions, using a bacterial strain of Acetobacter aceti var. xylinum. Use of agitated culture conditions results in sustained production, over an average of 70 hours, of at least 0.1 g/liter per hour of the desired cellulose. Wet cake reticulated cellulose, containing approximately 80-85% water, can be produced using the methods and conditions disclosed in the above-mentioned patents. Dry reticulated bacterial cellulose can be produced using drying techniques, such as spray-drying or freeze-drying, that are well known. See U.S. Pat. Nos. 5,079,162 and 5,144,021.

Acetobacter is characteristically a gram-negative, rod shaped bacterium 0.6-0.8 microns by 1.0-4 microns. It is a strictly aerobic organism; that is, metabolism is respiratory, not fermentative. This bacterium is further distinguished by the ability to produce multiple poly β-1,4-glucan chains, chemically identical to cellulose. The microcellulose chains, or microfibers, of reticulated bacterial cellulose are synthesized at the bacterial surface, at sites external to the cell membrane. These microfibers have a cross sectional dimensions of about 1.6 nm to about 3.2 nm by about 5.8 nm to about 133 nm. In one embodiment, the bacterial cellulose network has a widest cross sectional microfiber width of from about 1.6 nm to about 200 nm, alternatively less than about 133 nm, alternatively less than about 100 nm, alternatively less than about 5.8 nm. Additionally, the bacterial cellulose network has an average microfiber length of at least 100 nm, alternatively from about 100 to about 1500 nm. In one embodiment, the bacterial cellulose network has a microfiber aspect ratio, meaning the average microfiber length divided by the widest cross sectional microfiber width, of from about 10:1 to about 1000:1, alternatively from about 100:1 to about 400:1, alternatively from about 200:1 to about 300:1.

The presence of the bacterial cellulose network can be detected by a STEM micrograph imaging. A liquid detergent composition sample is obtained. A 1500 mesh copper TEM grid is placed on filter paper and 15 drops of the sample are applied to the TEM grid. The TEM grid is transferred to fresh filter paper and rinsed with 15 drops of deionized water. The TEM grid is then imaged in a S-5200 STEM micrograph instrument to observe for a fibrous network. Those of skill in the art will understand that if a fibrous network is detected, the cross dimensional of the fibers as well as the aspect ratio can be determined. Those of skill in the art will also recognized that alternative analytic techniques can be used to detect the presence of the bacterial cellulose network such as Atomic Force Microscopy using the same TEM grid and deposition and rinsing steps as disclosed above. An Atomic Force Microscopy 3D representation can be obtained showing the fiber dimensions as well as degree of networking.

The small cross sectional size of these Acetobacter-produced fibers, together with the large length and the inherent hydrophilicity of cellulose, provides a cellulose product having an unusually high capacity for absorbing aqueous solutions. Additives have often been used in combination with the bacterial cellulose to aid in the formation of stable, viscous dispersions. Non-limiting examples of additional suitable bacterial celluloses are disclosed in and U.S. Pat. Nos. 6,967,027 to Heux et al.; 5,207,826 to Westland et al.; 4,487,634 to Turbak et al.; 4,373,702 to Turbak et al. and 4,863,565 to Johnson et al., U.S. Pat. Publ. No. 2007/0027108 to Yang et al.

i. Methods of Activating the Bacterial Cellulose

In one embodiment, the bacterial cellulose network is formed by activating the bacterial cellulose under intense high shear processing conditions. It has importantly been found that the use of intense high shear processing conditions provides the bacterial cellulose network with enhanced structuring capabilities. By using intense processing conditions, the bacterial cellulose network can provide the desired structuring benefits at lower levels and without a need for costly chemical and physical modifications.

In one embodiment, the step of activating said bacterial cellulose under intense high shear processing conditions comprises: activating the bacterial cellulose and a solvent, e.g. water, at an energy density above about 1.0×10⁶ J/m³, alternatively above than 2.0×10⁶ J/m³. In one embodiment, the step of activation is performed with an energy density from 2.0×10⁶ J/m³ to about 5.0×10⁷ J/m³, alternatively from about 5.0×10⁶ J/m³ to about 2.0×10⁷ J/m³, alternatively from about 8.0×10⁶ J/m³ to about 1.0×10⁷ J/m³.

Processing techniques capable of providing this amount of energy density include conventional high shear mixers, static mixers, prop and in-tank mixers, rotor-stator mixers, and Gaulin homogenizers, and SONOLATOR® from Sonic Corp of CT. In one embodiment, the step of activating the bacterial cellulose comprising is performed with a high pressure homogenizer comprising a mixing chamber and a vibrating blade, wherein the feed is forced into the mixing chamber through an orifice. The feed which is under pressure accelerates as it passes through the orifice and comes into contact with the vibrating blade.

In one embodiment, the step of activating said bacterial cellulose under intense high shear processing conditions involves causing hydrodynamic cavitation is achieved using a SONOLATOR®. Without intending to be bound by theory, it is believed that the mixture within the mixing chamber undergoes hydrodynamic cavitation within the mixing chamber causing the bacterial cellulose to form a bacterial cellulose network with sufficient degree of interconnectivity to provide enhanced shear thinning capabilities. In one embodiment, the step of activating said bacterial cellulose in the liquid matrix is performed as a premix before the liquid matrix is combined with the encapsulated active portion of the composition. In another embodiment, the step of activation is performed on the liquid matrix and the encapsulated active concurrently in the same mixing chamber.

ii. Polymeric Thickener Coated Bacterial Cellulose

In one embodiment, the external structuring system further comprises a bacterial cellulose which is at least partially coated with a polymeric thickener. This at least partially coated bacterial cellulose can be prepared in accordance with the methods disclosed in U.S. Pat. Publ. No. 2007/0027108 to Yang et al. at ¶¶ 8-19. In one suitable process, the bacterial cellulose is subjected to mixing with a polymeric thickener to at least partially coat the bacterial cellulose fibers and bundles. It is believed that the commingling of the bacterial cellulose and the polymeric thickener allows for the desired generation of a polymeric thickener coating on at least a portion of the bacterial cellulose fibers and/or bundles.

In one embodiment the method of producing said at least partially coated bacterial cellulose comprises a proportion of bacterial cellulose to polymeric thickener comprises from about 0.1% to about 5% of the bacterial cellulose, alternatively from about 0.5% to about 3.0%, by weight of the added polymeric thickener; and from about 10% to about 900% of the polymeric thickener by weight of the bacterial cellulose.

In one embodiment the polymeric thickener comprises a hydrocolloid, at least on charged cellulose ether, at least one polymeric gum, and mixtures thereof. One suitable hydrocolloid includes carboxymethylcellulose (“CMC”). Suitable polymeric gums comprises xanthan products, pectin, alginates, gellan gum, welan gum, diutan gum, rhamsan gum, kargeenan, guar gum, agar, gum Arabic, gum ghatti, karay gum, gum tragacanth, tamarind gum, locust bean gum, and the like and mixtures there. See U.S. Pat. Publ. No. 2007/0027108 at ¶¶ 6 and 16.

In another embodiment, the bacterial cellulose undergoes no further modified either chemically or physically aside from the activation and/or the polymeric thickener coating. In one embodiment, the bacterial cellulose is free of a chemical modification comprising esterification or etherification by the addition of hydrophobic groups onto the fibers, meaning that the bacterial cellulose fibers are not modified to be surface active, wherein surface active means the ingredient lowers the surface tension of the medium in which it is dissolved. In another embodiment, the bacterial cellulose is free of any physical modification including coating the fibers with hydrophobic materials. It has importantly been found that by activating the bacterial cellulose network in accordance with the invention herein, the fibers do not need to be modified as mentioned in WO Publication No. 2007/068344 to Cai et al.

b. Non-Polymeric Crystalline Hydroxyl-Functional Materials

The fibrous network structurant of the present invention can also be made up of one or more non-polymeric crystalline hydroxyl-functional materials which include hydrophobically modified compositions such as hydrogenated castor oil. One suitable additional structuring agent comprises a non-polymeric (except for conventional alkyoxlation), crystalline hydroxyl-functional materials, which forms thread-like structuring systems throughout the liquid matrix when they are crystallized within the matrix in situ. Such materials can be generally characterized as crystalline, hydroxyl-containing fatty acids, fatty esters or fatty waxes. See e.g. U.S. Pat. No. 7,169,741 at col. 9, line 61 to col. 11, line 4, and 6,080,708 and in WO Publ. No. 2002/0040627, and US Patent Publ. 2005/0203213 at paragraphs 72-89.

c. Additional Structuring Agents

In another embodiment, the external structuring system further comprises from about 0.01% to about 5%, alternatively from about 0.1% to about 1%, alternatively up to about 2%, alternatively up to about 3% of an additional structuring agents such as, polymeric structuring agents, polymer gums, and mixtures thereof. Alternatively, the additional structuring agent can be incorporated at a low level, such as below 1%, below about 0.5%, below about 0.1%, below about 0.05% so that it still provides some structuring benefits without resulting in undesirable compositional incompatibility such as described above.

Other types of external structuring agents which may be utilized in the present compositions include polymer and/or gum structurants. Polymeric materials which will provide shear-thinning capabilities to the liquid matrix may also be employed. Suitable polymeric structuring agents include those of the polyacrylate, polysaccharide or polysaccharide derivative type. Polysaccharide derivatives typically used as structuring agents comprise polymeric gum materials. Such gums include pectine, alginate, arabinogalactan (gum Arabic), carrageenan, gellan gum, xanthan gum and guar gum. Gellan gum is a heteropolysaccharide prepared by fermentation of Pseudomonaselodea ATCC 31461 and is commercially marketed by CP Kelco U.S., Inc. under the KELCOGEL tradename. Processes for preparing gellan gum are described in U.S. Pat. Nos. 4,326,052; 4,326,053; 4,377,636 and 4,385,123.

In one embodiment, the external structuring system is free of essentially free of either one of the fibrous network structurants and/or any additional structuring agent known in the art such as those listed herein, for example: free or essentially free of non-polymeric crystalline hydroxyl-functional materials; free or essentially free of polymeric structuring agents including polymeric gums, pectine, alginate, arabinogalactan (gum Arabic), carrageenan, gellan gum, xanthan gum and guar gum. It has importantly been found that the external structuring system of the present invention provides sufficient rheological benefits, such as bead suspension and shear thinning capabilities, without reliance on structuring ingredients beyond the bacterial cellulose network described herein.

2. Encapsulated Active

### Encapsulated Active

In one embodiment, the encapsulated benefit agent has a certain combination of physical and chemical characteristics as defined by the following parameters: particle size coefficient of variation, fracture strength, benefit agent retention ratio and average particle size. Such parameters may be combined to yield a Delivery Index.

In one aspect, Applicants' particle comprises a core material and a wall material that at least partially surrounds the core material, said particle having a Delivery Index of at least about 0.05, at least about 7, or at least about 70.

In one aspect, Applicants' particle comprises a core material and a wall material that at least partially surrounds the core material, said particle having:
a.) a particle size coefficient of variation of from about 1.5 to about 6.0, from about 2.0 to about 3.5, or even from about 2.5 to about 3.2;
b.) a fracture strength of from about 0.1 psia to about 110 psia, from about 1 to about 50 psia, or even from about 4 to about 16 psia;
c.) a benefit agent retention ratio of from about 2 to about 110, from about 30 to about 90, or even from about 40 to about 70; and
d.) an average particle size of from about 1 micron to about 100 microns, from about 5 microns to about 80 microns, or even from about 15 microns to about 50 microns.

In one aspect of Applicants' invention, said particle may have and/or comprise any combination of the parameters described in the present specification.

Useful wall materials include materials selected from the group consisting of polyethylenes, polyamides, polystyrenes, polyisoprenes, polycarbonates, polyesters, polyacrylates, polyureas, polyurethanes, polyolefins, polysaccharides, epoxy resins, vinyl polymers, and mixtures thereof. In one aspect, useful wall materials include materials that are sufficiently impervious to the core material and the materials in the environment in which the encapsulated active will be employed, to permit the delivery benefit to be obtained. Suitable impervious wall materials include materials selected from the group consisting of reaction products of one or more amines with one or more aldehydes, such as urea cross-linked with formaldehyde or gluteraldehyde, melamine cross-linked with formaldehyde; gelatin-polyphosphate coacervates optionally cross-linked with gluteraldehyde; gelatin-gum Arabic coacervates; cross-linked silicone fluids; polyamine reacted with polyisocyanates and mixtures thereof. In one aspect, the wall material comprises melamine cross-linked with formaldehyde.

Useful core materials include perfume raw materials, silicone oils, waxes, hydrocarbons, higher fatty acids, essential oils, lipids, skin coolants, vitamins, sunscreens, antioxidants, glycerine, catalysts, bleach particles, silicon dioxide particles, malodor reducing agents, dyes, brighteners, antibacterial actives, antiperspirant actives, cationic polymers and mixtures thereof. In one aspect, said perfume raw material is selected from the group consisting of alcohols, ketones, aldehydes, esters, ethers, nitriles alkenes. In one aspect the core material comprises a perfume. In one aspect, said perfume comprises perfume raw materials selected from the group consisting of alcohols, ketones, aldehydes, esters, ethers, nitriles alkenes and mixtures thereof. In one aspect, said perfume may comprise a perfume raw material selected from the group consisting of perfume raw materials having a boiling point (B.P.) lower than about 250° C. and a ClogP lower than about 3, perfume raw materials having a B.P. of greater than about 250° C. and a ClogP of greater than about 3, perfume raw materials having a B.P. of greater than about 250° C. and a ClogP lower than about 3, perfume raw materials having a B.P. lower than about 250° C. and a ClogP greater than about 3 and mixtures thereof. Perfume raw materials having a boiling point B.P. lower than about 250° C. and a ClogP lower than about 3 are known as Quadrant I perfume raw materials, perfume raw materials having a B.P. of greater than about 250° C. and a ClogP of greater than about 3 are known as Quadrant IV perfume raw materials, perfume raw materials having a B.P. of greater than about 250° C. and a ClogP lower than about 3 are known as Quadrant II perfume raw materials, perfume raw materials having a B.P. lower than about 250° C. and a ClogP greater than about 3 are known as a Quadrant III perfume raw materials. In one aspect, said perfume comprises a perfume raw material having B.P. of lower than about 250° C. In one aspect, said perfume comprises a perfume raw material selected from the group consisting of Quadrant I, II, III perfume raw materials and mixtures thereof. In one aspect, said perfume comprises a Quadrant III perfume raw material. Suitable Quadrant I, II, III and IV perfume raw materials are disclosed in U.S. Pat. No. 6,869,923 B1.

In one aspect, said perfume comprises a Quadrant IV perfume raw material. While not being bound by theory, it is believed that such Quadrant IV perfume raw materials can improve perfume odor “balance”. Said perfume may comprise, based on total perfume weight, less than about 30%, less than about 20%, or even less than about 15% of said Quadrant IV perfume raw material.

The perfume raw materials and accords may be obtained from one or more of the following companies Firmenich (Geneva, Switzerland), Givaudan (Argenteuil, France), IFF (Hazlet, N.J.), Quest (Mount Olive, N.J.), Bedoukian (Danbury, Conn.), Sigma Aldrich (St. Louis, Mo.), Millennium Specialty Chemicals (Olympia Fields, Ill.), Polarone International (Jersey City, N.J.), Fragrance Resources (Keyport, N.J.), and Aroma & Flavor Specialties (Danbury, Conn.).

### Process of Making Encapsulated Actives

The particle disclosed in the present application may be made via the teachings of U.S. Pat. No. 6,592,990 B2 and/or U.S. Pat. No. 6,544,926 B1 and the examples disclosed herein.

Anionic emulsifiers are typically used during the capsule making process to emulsify the active prior to microcapsule formation. While not being bound by theory, it is believed that the anionic materials adversely interact with the cationic surfactant actives that are often found in compositions such as fabric care compositions—this may yield an aesthetically unpleasing aggregation of particles that are employed in said composition. In addition to the unacceptable aesthetics, such aggregates may result in rapid phase separation of the particles from the bulk phase. Applicants discovered that such aggregates can be prevented by the addition of certain aggregate inhibiting materials including materials selected from the group consisting of salts, polymers and mixtures thereof. Useful aggregate inhibiting materials include, divalent salts such as magnesium salts, for example, magnesium chloride, magnesium acetate, magnesium phosphate, magnesium formate, magnesium boride, magnesium titanate, magnesium sulfate heptahydrate; calcium salts, for example, calcium chloride, calcium formate, calcium calcium acetate, calcium bromide; trivalent salts, such as aluminum salts, for example, aluminum sulfate, aluminum phosphate, aluminum chloride n-hydrate and polymers that have the ability to suspend anionic particles such as soil suspension polymers, for example, polyamines (polyethylene imines, alkoxylated polyethylene imines, polyquaternium-6 and polyquaternium-7.

In one aspect, Calcium Formate and/or formic acid may be added to an aqueous slurry of microcapsules, for example, perfume microcapsules. Calcium Formate and/or formic acid is typically combined with, based on total slurry weight, at a level of from about 0.6 wt % to about 3 wt. %, from about 1 wt % to about 2 wt. % or even from about 1.2 wt % to about 1.5 wt. %, said microcapsule slurry. Calcium Formate and/or formic acid may provide the following benefits: slurry phase separation inhibition, aggregate formation inhibition and microbial inhibition. Typically, the aforementioned microbial inhibition is achieved when the slurry and/or product comprising said slurry has a pH of 3.8 or less. Calcium Formate may be obtained from Perstorp Inc., of Toledo, Ohio U.S.A. and formic acid may be obtained from Aldrich, P.O. Box 2060, Milwaukee, Wis. 53201, USA.

In one aspect of the invention, encapsulated actives are manufactured and are subsequently coated with a material to reduce the rate of leakage of the active from the particles when the particles are subjected to a bulk environment containing, for example, surfactants, polymers, and solvents. Non-limiting examples of coating materials that can serve as barrier materials include materials selected from the group consisting of polyvinyl pyrrolidone homopolymer, and its various copolymers with styrene, vinyl acetate, imidazole, primary and secondary amine containing monomers, methyl acrylate, polyvinyl acetal, maleic anhydride; polyvinyl alcohol homopolymer, and its various copolymers with vinyl acetate, 2-acrylamide-2-methylpropane sulfonate, primary and secondary amine containing monomers, imidazoles, methyl acrylate; polyacrylamides; polyacrylic acids; microcrystalline waxes; paraffin waxes; modified polysaccharides such as waxy maize or dent corn starch, octenyl succinated starches, derivatized starches such as hydroxyethylated or hydroxypropylated starches, carrageenan, guar gum, pectin, xanthan gum; modified celluloses such as hydrolyzed cellulose acetate, hydroxy propyl cellulose, methyl cellulose, and the like; modified proteins such as gelatin; hydrogenated and non-hydrogenated polyalkenes; fatty acids; hardened shells such as urea crosslinked with formaldehyde, gelatin-polyphosphate, melamine-formaldehyde, polyvinyl alcohol crosslinked with sodium tetraborate or gluteraldehyde; latexes of styrene-butadiene, ethyl cellulose, inorganic materials such as clays including magnesium silicates, aluminosilicates; sodium silicates, and the like; and mixtures thereof. Such materials can be obtained from CP Kelco Corp. of San Diego, Calif., USA; Degussa AG or Dusseldorf, Germany; BASF AG of Ludwigshafen, Germany; Rhodia Corp. of Cranbury, N.J., USA; Baker Hughes Corp. of Houston, Tex., USA; Hercules Corp. of Wilmington, Del., USA; Agrium Inc. of Calgary, Alberta, Canada, ISP of New Jeresy U.S.A.

In one aspect wherein the particle is employed in a fabric conditioning composition, the coating material comprises sodium silicate. While not being bound by theory, it is believed that sodium silicate's solubility at high pH, but poor solubility at low pH makes it an ideal material for use on particles that may be used in compositions that are formulated at pH below 7 but used in an environment wherein the pH is greater or equal to 7. The encapsulated actives made be made by following the procedure described in U.S. Pat. No. 6,592,990. However, the coating aspect of the present invention is not limited to the encapsulated actives of the present invention as any encapsulated active may benefit from the coatings and coating processes disclosed herein.

Suitable equipment for use in the processes disclosed herein may include continuous stirred tank reactors, homogenizers, turbine agitators, recirculating pumps, paddle mixers, ploughshear mixers, ribbon blenders, vertical axis granulators and drum mixers, both in batch and, where available, in continuous process configurations, spray dryers, and extruders. Such equipment can be obtained from Lodige GmbH (Paderborn, Germany), Littleford Day, Inc. (Florence, Ky., U.S.A.), Forberg AS (Larvik, Norway), Glatt Ingenieurtechnik GmbH (Weimar, Germany), Niro (Soeborg, Denmark), Hosokawa Bepex Corp. (Minneapolis, Minn., USA), Arde Barinco (New Jersey, USA).

### Formaldehyde Scavenging

In one aspect, encapsulated actives may be combined with a formaldehyde scavenger. In one aspect, such encapsulated actives may comprise the encapsulated actives of the present invention. Suitable formaldehyde scavengers include materials selected from the group consisting of sodium bisulfite, urea, ethylene urea, cysteine, cysteamine, lysine, glycine, serine, carnosine, histidine, glutathione, 3,4-diaminobenzoic acid, allantoin, glycouril, anthranilic acid, methyl anthranilate, methyl 4-aminobenzoate, ethyl acetoacetate, acetoacetamide, malonamide, ascorbic acid, 1,3-dihydroxyacetone dimer, biuret, oxamide, benzoguanamine, pyroglutamic acid, pyrogallol, methyl gallate, ethyl gallate, propyl gallate, triethanol amine, succinamide, thiabendazole, benzotriazol, triazole, indoline, sulfanilic acid, oxamide, sorbitol, glucose, cellulose, poly(vinyl alcohol), partially hydrolyzed poly(vinylformamide), poly(vinyl amine), poly(ethylene imine), poly(oxyalkyleneamine), poly(vinyl alcohol)-co-poly(vinyl amine), poly(4-aminostyrene), poly(1-lysine), chitosan, hexane diol, ethylenediamine-N,N′-bisacetoacetamide, N-(2-ethylhexyl)acetoacetamide, 2-benzoylacetoacetamide, N-(3-phenylpropyl)acetoacetamide, lilial, helional, melonal, triplal, 5,5-dimethyl-1,3-cyclohexanedione, 2,4-dimethyl-3-cyclohexanecarboxaldehyde, 2,2-dimethyl-1,3-dioxan-4,6-dione, 2-pentanone, dibutyl amine, triethylenetetramine, ammonium hydroxide, benzylamine, hydroxycitronellal, cyclohexanone, 2-butanone, pentane dione, dehydroacetic acid, or a mixture thereof. These formaldehyde scavengers may be obtained from Sigma/Aldrich/Fluka of St. Louis, Mo. U.S.A. or PolySciences, Inc. of Warrington, Pa. U.S.A.

Such formaldehyde scavengers are typically combined with a slurry containing said encapsulated active, at a level, based on total slurry weight, of from about 2 wt. % to about 18 wt. %, from about 3.5 wt. % to about 14 wt. % or even from about 5 wt. % to about 13 wt. %.

In one aspect, such formaldehyde scavengers may be combined with a product containing a encapsulated active, said scavengers being combined with said product at a level, based on total product weight, of from about 0.005% to about 0.8%, alternatively from about 0.03% to about 0.5%, alternatively from about 0.065% to about 0.25% of the product formulation.

In another aspect, such formaldehyde scavengers may be combined with a slurry containing said encapsulated active, at a level, based on total slurry weight, of from about 2 wt. % to about 14 wt. %, from about 3.5 wt. % to about 14 wt. % or even from about 5 wt. % to about 14 wt. % and said slurry may be added to a product matrix to which addition an identical or different scavenger may be added at a level, based on total product weight, of from about 0.005% to about 0.5%, alternatively from about 0.01% to about 0.25%, alternatively from about 0.05% to about 0.15% of the product formulation,

In one aspect, one or more of the aforementioned formaldehyde scavengers may be combined with a liquid fabric enhancing product containing a encapsulated active at a level, based on total liquid fabric enhancing product weight, of from 0.005% to about 0.8%, alternatively from about 0.03% to about 0.4%, alternatively from about 0.06% to about 0.25% of the product formulation

In one aspect, such formaldehyde scavengers may be combined with a liquid laundry detergent product containing a encapsulated active, said scavengers being selected from the group consisting of sodium bisulfite, urea, ethylene urea, cysteine, cysteamine, lysine, glycine, serine, carnosine, histidine, glutathione, 3,4-diaminobenzoic acid, allantoin, glycouril, anthranilic acid, methyl anthranilate, methyl 4-aminobenzoate, ethyl acetoacetate, acetoacetamide, malonamide, ascorbic acid, 1,3-dihydroxyacetone dimer, biuret, oxamide, benzoguanamine, pyroglutamic acid, pyrogallol, methyl gallate, ethyl gallate, propyl gallate, triethanol amine, succinamide, thiabendazole, benzotriazol, triazole, indoline, sulfanilic acid, oxamide, sorbitol, glucose, cellulose, poly(vinyl alcohol), partially hydrolyzed poly(vinylformamide), poly(vinyl amine), poly(ethylene imine), poly(oxyalkyleneamine), poly(vinyl alcohol)-co-poly(vinyl amine), poly(4-aminostyrene), poly(1-lysine), chitosan, hexane diol, ethylenediamine-N,N′-bisacetoacetamide, N-(2-ethylhexyl)acetoacetamide, 2-benzoylacetoacetamide, N-(3-phenylpropyl)acetoacetamide, lilial, helional, melonal, triplal, 5,5-dimethyl-1,3-cyclohexanedione, 2,4-dimethyl-3-cyclohexanecarboxaldehyde, 2,2-dimethyl-1,3-dioxan-4,6-dione, 2-pentanone, dibutyl amine, triethylenetetramine, ammonium hydroxide, benzylamine, hydroxycitronellal, cyclohexanone, 2-butanone, pentane dione, dehydroacetic acid and mixtures thereof, and combined with said liquid laundry detergent product at a level, based on total liquid laundry detergent product weight, of from about 0.003 wt. % to about 0.20 wt. %, from about 0.03 wt. % to about 0.20 wt. % or even from about 0.06 wt. % to about 0.14 wt. %.

In one aspect, such formaldehyde scavengers may be combined with a hair conditioning product containing a encapsulated active, at a level, based on total hair conditioning product weight, of from about 0.003 wt. % to about 0.30 wt. %, from about 0.03 wt. % to about 0.20 wt. % or even from about 0.06 wt. % to about 0.14 wt. %., said selection of scavengers being identical to the list of scavengers in the previous paragraph relating to a liquid laundry detergent product.

### Salt

In one embodiment, the composition further comprises salt at a level of from about 0% to about 4%, alternatively at least 1 about %, alternatively at least about 2%, alternatively at least about 2.5% by weight of said composition. In another embodiment, the composition is free or essentially free of a salt. Importantly, it is now believed that the addition of the fibrous network structurant provides an external structurant which is capable of providing the desired rheology to the composition without excessive compositional incompatibility in the presence of the salt and/or pH. Further, the desired rheology benefits include encapsulated active suspension and distribution throughout the composition as well as shear thickening benefits. Non-limiting examples of suitable salts include any salts used in encapsulated active slurry compositions which provides thickening or other desired compositional benefits.

The compositions of the present invention may also comprise, one or more electrolytes for control of phase stability, viscosity, and/or clarity. For example, the presence of certain electrolytes inter alia calcium chloride, magnesium chloride may be key to insuring initial product clarity and low viscosity, or may affect the dilution viscosity of liquid embodiments, especially isotropic liquid embodiments. Not wishing to be limited by theory, but only wishing to provide an example of a circumstance wherein the formulator must insure proper dilution viscosity, includes the following example. An electrolyte may be added to the compositions of the present invention to insure phase stability and prevent the fabric modifying compound from “gelling out” or from undergoing an undesirable or unacceptable viscosity increase. Prevention of gelling or formation of a “swelled”, high viscosity solution insures thorough delivery of the fabric modifying composition during the process of the present invention.

Those skilled in the art will recognize, however, that the level of electrolyte is also influenced by other factors inter alia the type of surface (i.e. fabric) onto which the composition is deposed and the final pH of the solution, the amount of principal solvent, and the level and type of any surfactants and other ingredients in the composition. Therefore, the formulator must consider all of the ingredients, namely, were the composition is a fabric treatment composition, the fabric treatment active, nonionic surfactant, and in the case of isotropic liquids, the principal solvent type and level, as well as level and identity of adjunct ingredients before selecting the type and/or level of electrolyte.

A wide variety of ionizable salts can be used. Examples of suitable salts are the halides of the Group IA and IIA metals of the Periodic Table, e.g., calcium chloride, sodium chloride, potassium bromide, and lithium chloride. The ionizable salts are particularly useful during the process of mixing the ingredients to make the compositions herein, and later to obtain the desired viscosity. The amount of ionizable salts used depends on the amount of active ingredients used in the compositions and can be adjusted according to the desires of the formulator. Typical levels of salts used to control the composition viscosity are from about 20 to about 10,000 parts per million (ppm), preferably from about 20 to about 5,000 ppm, of the composition.

Alkylene polyammonium salts can be incorporated into the composition to give viscosity control in addition to or in place of the water-soluble, ionizable salts above, In addition, these agents can act as scavengers, forming ion pairs with anionic detergent carried over from the main wash, in the rinse, and on the fabrics, and can improve softness performance. These agents can stabilized the viscosity over a broader range of temperature, especially at low temperatures, compared to the inorganic electrolytes. Specific examples of alkylene polyammonium salts include L-lysine, monohydrochloride and 1,5-diammonium 2-methyl pentane dihydrochloride.

3. Compositions Comprising Encapsulated Active

In one aspect, said composition is a consumer product. It should be understood that the composition comprising the encapsulated active can be a consumer product in itself without any of the following additional actives. In one embodiment, the structured composition is free or essentially free of any of the additional actives described in this sub-section. While the precise level of particle that is employed depends on the type and end use of the composition, a composition may comprise from about 0.01 to about 10, from about 0.1 to about 10, or even from about 0.2 to about 5 weight % of said encapsulated active based on total composition weight. In one aspect, a cleaning composition may comprise, from about 0.1 to about 1 weight % of such encapsulated active based on total cleaning composition weight of such encapsulated active. In one aspect, a fabric treatment composition may comprise, based on total fabric treatment composition weight, form about 0.01 to about 10% of such encapsulated active.

Aspects of the invention include the use of the encapsulated actives of the present invention in laundry detergent compositions (e.g., TIDE™), hard surface cleaners (e.g., MR CLEAN™), automatic dishwashing liquids (e.g., CASCADE™), dishwashing liquids (e.g., DAWN™), floor cleaners (e.g., SWIFFER™), a fabric softening composition (e.g. DOWNY™) an air freshening or textile freshener (e.g. FEBREZE™). Non-limiting examples of cleaning compositions may include those described in U.S. Pat. Nos. 4,515,705; 4,537,706; 4,537,707; 4,550,862; 4,561,998; 4,597,898; 4,968,451; 5,565,145; 5,929,022; 6,294,514; and 6,376,445. The cleaning compositions disclosed herein are typically formulated such that, during use in aqueous cleaning operations, the wash water will have a pH of between about 6.5 and about 12, or between about 7.5 and 10.5. Liquid dishwashing product formulations typically have a pH between about 6.8 and about 9.0. Cleaning products are typically formulated to have a pH of from about 7 to about 12. Techniques for controlling pH at recommended usage levels include the use of buffers, alkalis, acids, etc., and are well known to those skilled in the art.

### Fabric Softening Compositions:

In one embodiment, the composition is used for fabric treatment purposes, said composition further comprising a fabric softening active (“FSA”). Suitable fabric softening actives, include, but are not limited to, quats, amines, fatty esters, sucrose esters, silicones, dispersible polyolefins, clays, polysaccharides, fatty oils, polymer latexes and mixtures thereof. Examples of these fabric softening actives, starches, silicones and other suitable adjunct ingredients are disclosed in US Patent Publ. 2007/0202063A1

Suitable quats include but are not limited to, materials selected from the group consisting of ester quats, amide quats, imidazoline quats, alkyl quats, amdioester quats and mixtures thereof. Suitable ester quats include but are not limited to, materials selected from the group consisting of monoester quats, diester quats, triester quats and mixtures thereof. Suitable amide quats include but are not limited to, materials selected from the group consisting of monoamide quats, diamide quats and mixtures thereof. Suitable alkyl quats include but are not limited to, materials selected from the group consisting of mono alkyl quats, dialkyl quats quats, trialkyl quats, tetraalkyl quats and mixtures thereof. Suitable amines include but are not limited to, materials selected from the group consisting of esteramines, amidoamines, imidazoline amines, alkyl amines, amdioester amines and mixtures thereof. Suitable ester amines include but are not limited to, materials selected from the group consisting of monoester amines, diester amines, triester amines and mixtures thereof. In one embodiment, the FSA is formed from a reaction product of a fatty acid and an aminoalcohol obtaining mixtures of mono-, di-, and, in one embodiment, triester compounds. In another embodiment, the FSA comprises one or more softener quaternary ammonium compounds such, but not limited to, as a monoalkyquaternary ammonium compound, dialkylquaternary ammonium compound, a diamido quaternary compound, a diester quaternary ammonium compound, or a combination thereof.

In one aspect, the FSA comprises a diester quaternary ammonium or protonated diester ammonium (hereinafter “DQA”) compound composition. In certain embodiments of the present invention, the DQA compound compositions also encompass diamido FSAs and FSAs with mixed amido and ester linkages as well as the aforementioned diester linkages, all herein referred to as DQA. One type of DQA (“DQA (1)”) suitable as a FSA in the present composition includes a compound comprising the formula:

{R₍₄₋ₘ₎—N⁺—[(CH₂)ₙ—Y—R¹]ₘ}X⁻

wherein each R substituent is either hydrogen, a short chain C₁-C₆, preferably C₁-C₃ alkyl or hydroxyalkyl group, e.g., methyl (most preferred), ethyl, propyl, hydroxyethyl, hydroxypropyl and the like, poly (C₂-C₃ alkoxy), preferably polyethoxy, benzyl, or mixtures thereof; each m is 2 or 3; each n is from 1 to about 4, preferably 2; each Y is —O—(O)C—, —C(O)—O—, —NR—C(O)—, or —C(O)—NR— and it is acceptable for each Y to be the same or different; the sum of carbons in each R¹, plus one when Y is —O—(O)C— or —NR—C(O)—, is C₁₂-C₂₂, preferably C₁₄-C₂₀, with each R¹ being a hydrocarbyl, or substituted hydrocarbyl group; it is acceptable for R¹ to be unsaturated or saturated and branched or linear and preferably it is linear; it is acceptable for each R¹ to be the same or different and preferably these are the same; and X⁻ can be any softener-compatible anion, preferably, chloride, bromide, methylsulfate, ethylsulfate, sulfate, phosphate, and nitrate, more preferably chloride or methyl sulfate.

In another aspect of the invention, the FSA comprises a compound, identified as DTDMAC comprising the formula:

[R₍₄₋ₘ₎—N⁺—R¹ₘ]A⁻

wherein each m is 2 or 3, each R¹ is a C₆-C₂₂, preferably C₁₄-C₂₀, but no more than one being less than about C₁₂ and then the other is at least about C₁₆, hydrocarbyl, or substituted hydrocarbyl substituent, preferably C₁₀-C₂₀ alkyl or alkenyl (unsaturated alkyl, including polyunsaturated alkyl, also referred to sometimes as “alkylene”), most preferably C₁₂-C₁₈ alkyl or alkenyl, and branched or unbranched. In one embodiment; each R is H or a short chain C₁-C₆, preferably C₁-C₃ alkyl or hydroxyalkyl group, e.g., methyl (most preferred), ethyl, propyl, hydroxyethyl, and the like, benzyl, or (R²O)₂₋₄H where each R² is a C₁-C₆ alkylene group; and A⁻ is a softener compatible anion, preferably, chloride, bromide, methylsulfate, ethylsulfate, sulfate, phosphate, or nitrate; more preferably chloride or methyl sulfate.

Additional suitable FSA's are described in U.S. Pat. Pub. No. 2004/0204337 A1, published Oct. 14, 2004 to Corona et al., from paragraphs 30-79, U.S. Pat. Pub. No. 2004/0229769 A1, published Nov. 18, 2005, to Smith et al., on paragraphs 26-31; or U.S. Pat. No. 6,494,920, at column 1, line 51 et seq. detailing an “esterquat” or a quaternized fatty acid triethanolamine ester salt.

Typical minimum levels of incorporation of the FSA in the present fabric care compositions are at least about 1%, alternatively at least about 2%, alternatively at least about at least about 3%, alternatively at least about at least about 5%, alternatively at least about 10%, and alternatively at least about 12%, by weight of the fabric care composition. The fabric care composition may typically comprise maximum levels of FSA of about less than about 90%, alternatively less than about 40%, alternatively less than about 30%, alternatively less than about 20%, by weight of the composition.

### Cationic Starch

One aspect of the invention provides a fabric softening composition comprising a cationic starch as a fabric softening active. In one embodiment, the fabric care compositions of the present invention generally comprise cationic starch at a level of from about 0.1% to about 7%, alternatively from about 0.1% to about 5%, alternatively from about 0.3% to about 3%, and alternatively from about 0.5% to about 2.0%, by weight of the composition. Cationic starch as a fabric softening active is described in U.S. Pat. Pub. 2004/0204337 A1, published Oct. 14, 2004, to Corona et al., at paragraphs 16-32. Suitable cationic starches for use in the present compositions are commercially-available from Cerestar under the trade name C*BOND® and from National Starch and Chemical Company under the trade name CATO® 2A.

### Silicone

In one embodiment, the fabric softening composition comprises a silicone. Suitable levels of silicone may comprise from about 0.1% to about 50%, alternatively from about 1% to about 40%, alternatively from about 2% to about 30%, alternatively from about 3% to about 20% by weight of the composition. Non limiting examples of silicones include those described in U.S. Pat. Pub. No. 2002/0077265 A1, to Buzzacarini et al., published Jun. 20, 2002 at paragraphs 51-57. Useful silicones can be any silicone comprising compound. In one embodiment, the silicone is a polydialkylsilicone, alternatively a polydimethyl silicone (polydimethyl siloxane or “PDMS”), or a derivative thereof. In another embodiment, the silicone is chosen from an aminofunctional silicone, alkyloxylated silicone, ethoxylated silicone, propoxylated silicone, ethoxylated/propoxylated silicone, quaternary silicone, or combinations thereof. Other useful silicone materials may include materials of the formula:

HO[Si(CH₃)₂—O]ₓ{Si(OH)[(CH₂)₃—NH—(CH₂)₂—NH₂]O}_{y}H

wherein x and y are integers which depend on the molecular weight of the silicone, preferably has a molecular weight such that the silicone exhibits a viscosity of from about 500 cSt to about 500,000 cSt at 25° C. This material is also known as “amodimethicone”.

### Other Fabric Softening Agents

In addition to or in lieu of fabric softening actives herein described, other materials can be used as fabric softening agents in compositions of the present invention. Non-limiting examples of these other agents include: clays, fatty oils, such as fatty acids, triglycerides, fatty alcohols, fatty esters, fatty amides, fatty amines; sucrose esters, dispersible polyethylenes, and polymer latexes. Examples of fatty acids are described in WO06007911A1 and WO06007899A1. Clays are described in U.S. Pat. Pub. No. 2004/0142841 A1 published Jul. 22, 2004, to de Buzzaccarini et al., from paragraphs 74-99.

Nonionic fabric care benefit agents can comprise sucrose esters, and are typically derived from sucrose and fatty acids. Sucrose ester is composed of a sucrose moiety having one or more of its hydroxyl groups esterified.

### Dispersible Polyolefins

Generally, all dispersible polyolefins that provide fabric care benefits can be used as water insoluble fabric care benefit agents in the present invention. The polyolefins can be in the format of waxes, emulsions, dispersions or suspensions. Non-limiting examples are discussed below.

In one embodiment, the polyolefin is chosen from a polyethylene, polypropylene, or a combination thereof. The polyolefin may be at least partially modified to contain various functional groups, such as carboxyl, alkylamide, sulfonic acid or amide groups. In another embodiment, the polyolefin is at least partially carboxyl modified or, in other words, oxidized.

For ease of formulation, the dispersible polyolefin may be introduced as a suspension or an emulsion of polyolefin dispersed by use of an emulsifying agent. The polyolefin suspension or emulsion preferably comprises from about 1% to about 60%, alternatively from about 10% to about 55%, alternatively from about 20% to about 50% by weight of polyolefin. The polyolefin preferably has a wax dropping point (see ASTM D3954-94, volume 15.04—“Standard Test Method for propping Point of Waxes”) from about 20° to about 170° C., alternatively from about 50° to about 140° C. Suitable polyethylene waxes are available commercially from suppliers including but not limited to Honeywell (A-C polyethylene), Clariant (Velustrol® emulsion), and BASF (LUWAX®).

When an emulsion is employed with the dispersible polyolefin, the emulsifier may be any suitable emulsification agent. Non-limiting examples include an anionic, cationic, nonionic surfactant, or a combination thereof. However, almost any suitable surfactant or suspending agent may be employed as the emulsification agent. The dispersible polyolefin is dispersed by use of an emulsification agent in a ratio to polyolefin wax of about 1:100 to about 1:2, alternatively from about 1:50 to about 1:5, respectively.

### Polymer Latexes

Polymer latex is made by an emulsion polymerization which includes one or more monomers, one or more emulsifiers, an initiator, and other components familiar to those of ordinary skill in the art. Generally, all polymer latexes that provide fabric care benefits can be used as water insoluble fabric care benefit agents of the present invention. Non-limiting examples of suitable polymer latexes include those disclosed in WO 02/18451; US 2004/0038851 A1; and US 2004/0065208 A1. Additional non-limiting examples include the monomers used in producing polymer latexes such as: (1) 100% or pure butylacrylate; (2) butylacrylate and butadiene mixtures with at least 20% (weight monomer ratio) of butylacrylate; (3) butylacrylate and less than 20% (weight monomer ratio) of other monomers excluding butadiene; (4) alkylacrylate with an alkyl carbon chain at or greater than C₆; (5) alkylacrylate with an alkyl carbon chain at or greater than C₆ and less than 50% (weight monomer ratio) of other monomers; (6) a third monomer (less than 20% weight monomer ratio) added into an aforementioned monomer systems; and (7) combinations thereof.

Polymer latexes that are suitable fabric care benefit agents in the present invention may include those having a glass transition temperature of from about −120° C. to about 120° C., alternatively from about −80° C. to about 60° C. Suitable emulsifiers include anionic, cationic, nonionic and amphoteric surfactants. Suitable initiators include initiators that are suitable for emulsion polymerization of polymer latexes. The particle size diameter (χ₅₀) of the polymer latexes can be from about 1 nm to about 10 μm, alternatively from about 10 nm to about 1 μm, preferably from about 10 nm to about 20 nm.

### Fatty Acid

One aspect of the invention provides a fabric softening composition comprising a fatty acid, preferably a free fatty acid. The term “fatty acid” is used herein in the broadest sense to include unprotonated or protonated forms of a fatty acid; and includes fatty acid that is bound or unbound to another chemical moiety as well as the various combinations of these species of fatty acid. One skilled in the art will appreciate that the pH of an aqueous composition will dictate, in part, whether a fatty acid is protonated or unprotonated. In another embodiment, the fatty acid is in its unprotonated, or salt form, together with a counter ion, such as, but not limited to, calcium, magnesium, sodium, potassium and the like. The term “free fatty acid” means a fatty acid that is not bound (to another chemical moiety (covalently or otherwise) to another chemical moiety.

In one embodiment, the fatty acid may include those containing from about 12 to about 25, preferably from about 13 to about 22, more preferably from about 16 to about 20, total carbon atoms, with the fatty moiety containing from about 10 to about 22, preferably from about 12 to about 18, more preferably from about 14 (mid-cut) to about 18 carbon atoms. Non-limiting examples of fatty acids (FA) are listed in U.S. Pat. No. 5,759,990 at col 4, lines 45-66. Mixtures of fatty acids from different fat sources can be used, and in some embodiments preferred.

The Iodine Value or “IV” measures the degree of unsaturation in the fatty acid. In one embodiment of the invention, the fatty acid has an IV preferably from about 40 to about 140, more preferably from about 50 to about 120 or from about 85 to about 105.

### Softening Oils

Another class of optional fabric care actives is softening oils, which include but are not limited to, vegetable oils (such as soybean, sunflower, and canola), hydrocarbon based oils (natural and synthetic petroleum lubricants, preferably polyolefins, isoparaffins, and cyclic paraffins), triolein, fatty esters, fatty alcohols, fatty amines, fatty amides, and fatty ester amines. Oils can be combined with fatty acid softening agents, clays, and silicones.

### Clays

In one embodiment of the invention, the fabric care composition may comprise a clay as a fabric care active. In one embodiment clay can be a softener or co-softeners with another softening active, for example, silicone. Preferred clays include those materials classified geologically smectites and are described in U.S. Pat. Appl. Publ. 20030216274 A1, to Valerio Del Duca, et al., published Nov. 20, 2003, paragraphs 107-120. Other suitable clays are described U.S. Pat. Nos. 3,862,058; 3,948,790; 3,954,632; 4,062,647; and U.S. Patent Appl. Pub. No. 20050020476A1 to Wahl, et. al., page 5, paragraph 0078 through page 6, paragraph 0087.

### Solvent

The compositions of the present invention will contain the suitable amounts of solvent in order to form the structured liquid matrix thereof. In one embodiment, the solvent is water. In one embodiment, solvent, such as water, comprises from about 0.1% to about 75%, alternatively from about 1% to about 30%, alternatively from about 10% to about 20%, or alternatively from about 30% to about 70%, alternatively from about 40% to about 60%, alternatively greater than about 50% by weight of the liquid detergent compositions herein. Those of skill in the art will understand that the solvent is measured as the solvent external to the capsule. Additional solvent can be present within the interior of the capsule when the active is a liquid.

### Adjunct Materials

While not essential for the purposes of the present invention, the non-limiting list of adjuncts illustrated hereinafter are suitable for use in the instant compositions and may be desirably incorporated in certain embodiments of the invention, for example to assist or enhance performance, for treatment of the substrate to be cleaned, or to modify the aesthetics of the composition as is the case with perfumes, colorants, dyes or the like. It is understood that such adjuncts are in addition to the components that are supplied via Applicants' delivery particles and FSAs. The precise nature of these additional components, and levels of incorporation thereof, will depend on the physical form of the composition and the nature of the operation for which it is to be used. Suitable adjunct materials include, but are not limited to, surfactants, builders, chelating agents, dye transfer inhibiting agents, dispersants, enzymes, and enzyme stabilizers, catalytic materials, bleach activators, polymeric dispersing agents, clay soil removal/anti-redeposition agents, brighteners, suds suppressors, dyes, additional perfume and perfume delivery systems, structure elasticizing agents, fabric softeners, carriers, hydrotropes, processing aids and/or pigments. In addition to the disclosure below, suitable examples of such other adjuncts and levels of use are found in U.S. Pat. Nos. 5,576,282, 6,306,812 B1 and 6,326,348 B1 that are incorporated by reference.

As stated, the adjunct ingredients are not essential to Applicants' cleaning and fabric care compositions. Thus, certain embodiments of Applicants' compositions do not contain one or more of the following adjuncts materials: bleach activators, surfactants, builders, chelating agents, dye transfer inhibiting agents, dispersants, enzymes, and enzyme stabilizers, catalytic metal complexes, polymeric dispersing agents, clay and soil removal/anti-redeposition agents, brighteners, suds suppressors, dyes, additional perfumes and perfume delivery systems, structure elasticizing agents, fabric softeners, carriers, hydrotropes, processing aids and/or pigments. However, when one or more adjuncts is present, such one or more adjuncts may be present as detailed below:

Surfactants—The compositions according to the present invention can comprise a surfactant or surfactant system wherein the surfactant can be selected from nonionic and/or anionic and/or cationic surfactants and/or ampholytic and/or zwitterionic and/or semi-polar nonionic surfactants. The surfactant is typically present at a level of from about 0.1%, from about 1%, or even from about 5% by weight of the cleaning compositions to about 99.9%, to about 80%, to about 35%, or even to about 30% by weight of the cleaning compositions. The liquid matrix comprises from about 0.01% to 70%, alternatively from about 1% to about 50%, alternatively from about 3% to about 20% of a surfactant system, by weight of the liquid detergent composition. The surfactant system of the present invention comprising: an anionic surfactant; a nonionic surfactant; a cationic surfactant; an ampholytic surfactant; a zwitterionic surfactant; and mixtures thereof. Suitable surfactants for use herein are disclosed in U.S. 2005/0203213 to Pommiers et al., 2004/0018950 to Foley et al., WO 2006/116099 to Fleckenstein et al., and U.S. Pat. No. 7,169,741 to Barry et al.

Suitable anionic surfactants include: alkali metal salts of C₁₀₋₁₆ alkyl benzene sulfonic acids, preferably C₁₁₋₁₄ alkyl benzene sulfonic acids; ethoxylated alkyl sulfate surfactants, such as a C₈-C₂₀ alkyl sulfate surfactant; unalkoyxylated, e.g., unethoxylated, alkyl ether sulfate surfactants are those produced by the sulfation of higher C₈-C₂₀ fatty alcohols; and mixtures thereof.

In another embodiment, the liquid matrix comprises from about 0.1% to about 20%, alternatively from about 0.2% to about 15%, alternatively from about 0.5% to about 10%, by weight of the liquid detergent composition, of a nonionic surfactant(s). Suitable nonionic surfactants include any of the conventional nonionic surfactant types typically used in liquid cleaning compositions. These include alkoxylated fatty alcohols, ethylene oxide (EO)-propylene oxide (PO) block polymers, and amine oxide surfactants. Suitable for use in the liquid cleaning compositions herein are those nonionic surfactants which are normally liquid.

In one embodiment, the liquid matrix comprises a weight ratio of surfactant system to external structurant, i.e. bacterial cellulose network, of from about 1:1 to about 5000:1, alternatively from about 100:1 to about 2000:1, alternatively from about 500:1 to about 1000:1.

Builders—The compositions of the present invention can comprise one or more detergent builders or builder systems. When present, the compositions will typically comprise at least about 1% builder, or from about 5% or 10% to about 80%, 50%, or even 30% by weight, of said builder. Builders include, but are not limited to, the alkali metal, ammonium and alkanolammonium salts of polyphosphates, alkali metal silicates, alkaline earth and alkali metal carbonates, aluminosilicate builders polycarboxylate compounds, ether hydroxypolycarboxylates, copolymers of maleic anhydride with ethylene or vinyl methyl ether, 1,3,5-trihydroxybenzene-2,4,6-trisulphonic acid, and carboxymethyl-oxysuccinic acid, the various alkali metal, ammonium and substituted ammonium salts of polyacetic acids such as ethylenediamine tetraacetic acid and nitrilotriacetic acid, as well as polycarboxylates such as mellitic acid, succinic acid, oxydisuccinic acid, polymaleic acid, benzene 1,3,5-tricarboxylic acid, carboxymethyloxysuccinic acid, and soluble salts thereof.

Chelating Agents—The compositions herein may also optionally contain one or more copper, iron and/or manganese chelating agents. If utilized, chelating agents will generally comprise from about 0.1% by weight of the compositions herein to about 15%, or even from about 3.0% to about 15% by weight of the compositions herein.

Dye Transfer Inhibiting Agents—The compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in the compositions herein, the dye transfer inhibiting agents are present at levels from about 0.0001%, from about 0.01%, from about 0.05% by weight of the cleaning compositions to about 10%, about 2%, or even about 1% by weight of the cleaning compositions.

Dispersants—The compositions of the present invention can also contain dispersants. Suitable water-soluble organic materials are the homo- or co-polymeric acids or their salts, in which the polycarboxylic acid may comprise at least two carboxyl radicals separated from each other by not more than two carbon atoms.

Enzymes—The compositions can comprise one or more detergent enzymes which provide cleaning performance and/or fabric care benefits. Examples of suitable enzymes include, but are not limited to, hemicellulases, peroxidases, proteases, cellulases, xylanases, lipases, phospholipases, esterases, cutinases, pectinases, keratanases, reductases, oxidases, phenoloxidases, lipoxygenases, ligninases, pullulanases, tannases, pentosanases, malanases, β-glucanases, arabinosidases, hyaluronidase, chondroitinase, laccase, and amylases, or mixtures thereof. A typical combination is a cocktail of conventional applicable enzymes like protease, lipase, cutinase and/or cellulase in conjunction with amylase.

Enzyme Stabilizers—Enzymes for use in compositions, for example, detergents can be stabilized by various techniques. The enzymes employed herein can be stabilized by the presence of water-soluble sources of calcium and/or magnesium ions in the finished compositions that provide such ions to the enzymes.

Catalytic Metal Complexes—Applicants' compositions may include catalytic metal complexes. One type of metal-containing bleach catalyst is a catalyst system comprising a transition metal cation of defined bleach catalytic activity, such as copper, iron, titanium, ruthenium, tungsten, molybdenum, or manganese cations, an auxiliary metal cation having little or no bleach catalytic activity, such as zinc or aluminum cations, and a sequestrate having defined stability constants for the catalytic and auxiliary metal cations, particularly ethylenediaminetetraacetic acid, ethylenediaminetetra (methyl-enephosphonic acid) and water-soluble salts thereof. Such catalysts are disclosed in U.S. Pat. No. 4,430,243.

If desired, the compositions herein can be catalyzed by means of a manganese compound. Such compounds and levels of use are well known in the art and include, for example, the manganese-based catalysts disclosed in U.S. Pat. No. 5,576,282. Cobalt bleach catalysts useful herein are known, and are described, for example, in U.S. Pat. Nos. 5,597,936 and 5,595,967. Such cobalt catalysts are readily prepared by known procedures, such as taught for example in U.S. Pat. Nos. 5,597,936, and 5,595,967.

Compositions herein may also suitably include a transition metal complex of a macropolycyclic rigid ligand—abreviated as “MRL”. As a practical matter, and not by way of limitation, the compositions and cleaning processes herein can be adjusted to provide on the order of at least one part per hundred million of the benefit agent MRL species in the aqueous washing medium, and may provide from about 0.005 ppm to about 25 ppm, from about 0.05 ppm to about 10 ppm, or even from about 0.1 ppm to about 5 ppm, of the MRL in the wash liquor. Preferred transition-metals in the instant transition-metal bleach catalyst include manganese, iron and chromium. Preferred MRL's herein are a special type of ultra-rigid ligand that is cross-bridged such as 5,12-diethyl-1,5,8,12-tetraazabicyclo[6.6.2]hexa-decane. Suitable transition metal MRLs are readily prepared by known procedures, such as taught for example in WO 00/32601, and U.S. Pat. No. 6,225,464.

### Processes of Making and Using Compositions

The compositions of the present invention can be formulated into any suitable form and prepared by any process chosen by the formulator, non-limiting examples of which are described in U.S. Pat. No. 5,879,584; U.S. Pat. No. 5,691,297; U.S. Pat. No. 5,574,005; U.S. Pat. No. 5,569,645; U.S. Pat. No. 5,565,422; U.S. Pat. No. 5,516,448; U.S. Pat. No. 5,489,392; U.S. Pat. No. 5,486,303 all of which are incorporated herein by reference.

4. Forms of the Composition and Methods of Use

It should be understood that the encapsulated slurry composition can be used in many different forms. These forms include as a raw material, as an additive, and as a consumer product which can be used in its immediate form. Those of skill in the art will understand that different forms can include different additional adjunct compositions based on the desired composition. In one embodiment where the composition is for use as a raw material, the amount of encapsulated active is from about 30% to about 70% of the encapsulated active by weight of the composition, alternatively from 40% to about 60%, alternatively from about 50% to about 65%. In another embodiment where the composition is an additive such as a laundry care additive, the amount of encapsulated active is from about 10% to about 30%, alternatively from about 15% to about 25%, alternatively from about 17% to about 22% by weight of the composition. In yet another embodiment where the composition is used as a consumer end product which can be consumed in its immediate form, the amount of encapsulated active is at a level of from about 0.5% to about 10%, alternatively from about 1% to about 5%, alternatively from about 2% to about 4% by weight of the composition.

Compositions containing the encapsulated active disclosed herein can be used as a raw material for later processing, as an additive such as a laundry additive, as a fragrance, a food additive, or any other suitable method in which there is a desire to encapsulate the active. Typically at least a portion of the situs is contacted with an embodiment of Applicants' composition, in neat form or diluted in a liquor, for example, a wash liquor and then the situs may be optionally washed and/or rinsed. In one aspect, a situs is optionally washed and/or rinsed, contacted with a particle according to the present invention or composition comprising said particle and then optionally washed and/or rinsed. For purposes of the present invention, washing includes but is not limited to, scrubbing, and mechanical agitation. The fabric may comprise most any fabric capable of being laundered or treated in normal consumer use conditions. Liquors that may comprise the disclosed compositions may have a pH of from about 3 to about 11.5. Such compositions are typically employed at concentrations of from about 500 ppm to about 15,000 ppm in solution. When the wash solvent is water, the water temperature typically ranges from about 5° C. to about 90° C. and, when the situs comprises a fabric, the water to fabric ratio is typically from about 1:1 to about 30:1.

### Test Methods

It is understood that the test methods that are disclosed in the Test Methods Section of the present application must be used to determine the respective values of the parameters of Applicants' invention as such invention is described and claimed herein.

(1) Particle size distribution
a.) Place 1 gram of particles in 1 liter of distilled deionized (DI) water.
b.) Permit the particles to remain in the DI water for 10 minutes and then recover the particles by filtration.
c.) Determine the particle size distribution of the particle sample by measuring the particle size of 50 individual particles using the experimental apparatus and method of Zhang, Z.; Sun, G; “Mechanical Properties of Melamine-Formaldehyde microcapsules,” J. Microencapsulation, vol 18, no. 5, pages 593-602, 2001.
d.) Average the 50 independent particle diameter measurements to obtain an average particle diameter.
e.) Use the 50 independent measurements to calculate a standard deviation of particle size using the following equation:

$$μ = \sqrt{\frac{\sum {( d - s )}^{2}}{n - 1}}$$

where
   μ is the standard deviation
   s is the average particle diameter
   d is the independent particle diameter
   n is the total number of particles whose diameter is measured.

(2) Active Retention Ratio
a.) Add 1 gram of particle to 99 grams of composition that the particle will be employed in.
b.) Age the particle containing composition of a.) above for 2 weeks at 40° C. in a sealed, glass jar.
c.) Recover the particles from b.) above by filtration.
d.) Treat the particles of c.) above with a solvent that will extract all the active out of the particle.
e.) Inject the active containing solvent from d.) above into a Gas Chromatograph and integrate the peak areas to determine the total quantity of active extracted from the particle sample.
f.) This quantity is then divided by the quantity that would be present if nothing had leaked out of the microcapsule (e.g. the total quantity of core material that is dosed into the composition via the microcapsules). This value is then multiplied by the ratio of average particle diameter to average particle thickness to obtain a Active Retention Ratio.
A detailed analytical procedure to measure the Active Retention Ratio is:

ISTD Solution

1. Weigh out 25 mg dodecane into a weigh boat.

2. Rinse the dodecane into a 1000 mL volumetric flask using ethanol.

3. Add ethanol to volume mark.

4. Stir solution until mixed. This solution is stable for 2 months.

Calibration Standard
1. Weigh out 75 mg of core material into a 100 mL volumetric flask.
2. Dilute to volume with ISTD solution to from above. This standard solution is stable for 2 months.
3. Mix well.
4. Analyze via GC/FID.

Basic Sample Prep

(Prepare Samples in Triplicate)
1. Weigh 1.000 gram sample of aged composition containing particles into a 100 mL tri-pour beaker. Record weight.
2. Add 4 drops (approximately 0.1 gram) 2-ethyl-1,3-Hexanediol into the tri-pour beaker.
3. Add 50 mL Deionized water to the beaker. Stir for 1 minute.
4. Using a 60 cc syringe, filter through a Millipore Nitrocellulose Filter Membrane (1.2 micron, 25 mm diameter).
5. Rinse through the filter with 10 mL of Hexane
6. Carefully remove the filter membrane and transfer to a 20 mL scintillation vial (using tweezers).
7. Add 10 mL ISTD solution (as prepared above) to the scintillation vial containing the filter.
8. Cap tightly, mix, and heat vial at 60° C. for 30 min.
9. Cool to room temperature.
10. Remove 1 mL and filter through a 0.45-micron PTFE syringe filter into GC vial. Several PTFE filters may be required to filter a 1 mL sample aliquot.
11. Analyze via GC/FID.

GG/FID Analysis Method:

Column—30 m×0.25 mm id, 1-um DB-1 phase

GC—6890 GC equipped with EPC control and constant flow capability

Method—50° C., 1 min. hold, temperature ramp of 4° C./min. to 300° C., and hold for 10 min.

Injector—1 uL splitless injection at 240° C.

GC/FID Analysis Method—Microbore Column Method:
Column—20 m×0.1 mm id, 0.1 μm DB-5
GC—6890 GC equipped with EPC control and constant flow capability (constant flow 0.4 mL/min)
Method—50° C., no hold, temperature ramp of 16° C./min to 275° C., and hold for 3 min.
Injector—1 μL split injection (80:1 split) at 250° C.

Calculations:

$$\% Total Perfume = \frac{{A}_{IS} \times {W}_{per - std} \times {A}_{per - sam}}{{A}_{per - std} \times {A}_{is - sam} \times {W}_{sam}} \times 100 \%$$

where
Aᵢₛ=Area of internal standard in the core material calibration standard;
W_{per-std}=weight of core material in the calibration sample
Aₚₑᵣ₋ₛₐₘ=Area of core material peaks in the composition containing particle sample;
A_{per-std}=Area of core material peaks in the calibration sample.
Aᵢₛ₋ₛₐₘ=Area of internal standard in composition containing particle sample;
Wₛₐₘ=Weight of the composition containing particle sample

$$Retention\_Ratio = ( \frac{Total\_Perfume}{Perfume\_Dosed \_Into \_Product \_Via \_Microcapsules} ) ( \frac{μ}{T} )$$

where
μ is the average particle diameter, from Test Method 1
T is the average particle thickness as calculated from Test Method 3

(3) Fracture Strength
a.) Place 1 gram of particles in 1 liter of distilled deionized (DI) water.
b.) Permit the particles to remain in the DI water for 10 minutes and then recover the particles by filtration.
c.) Determine the average rupture force of the particles by averaging the rupture force of 50 individual particles. The rupture force of a particle is determined using the procedure given in Zhang, Z.; Sun, G; “Mechanical Properties of Melamine-Formaldehyde microcapsules,” J. Microencapsulation, vol 18, no. 5, pages 593-602, 2001. Then calculate the average fracture pressure by dividing the average rupture force (in Newtons) by the average cross-sectional area (as determined by Test Method 1 above) of the spherical particle (πr², where r is the radius of the particle before compression).
d.) Calculate the average fracture strength by using the following equation:

$${σ}_{fracture\_stress} = \frac{P}{4 ( d / T )}$$

where
   P is the average fracture pressure from a.) above
   d is the average diameter of the particle (as determined by Test Method 1 above)
   T is the average shell thickness of the particle shell as determined by the following equation:

$$T = \frac{{r}_{capsule} ( 1 - c ) {ρ}_{perfume}}{3 [ c {ρ}_{wall} + ( 1 - c ) {ρ}_{perfume} ]}$$

where
   c is the average perfume content in the particle
   r is the average particle radius
   ρ_{wall} the average density of the shell as determined by ASTM method is method B923-02, “Standard Test Method for Metal Powder Skeletal Density by Helium or Nitrogen Pycnometry”, ASTM International.
   ρ_{perfume} is the average density of the perfume as determined by ASTM method D1480-93 (1997) “Standard Test Method for Density and Relative Density (Specific Gravity) of Viscous Materials by Bingham Pycnometer”, ASTM International.

(4) ClogP
The “calculated logP” (ClogP) is determined by the fragment approach of Hansch and Leo (cf., A. Leo, in Comprehensive Medicinal Chemistry, Vol. 4, C. Hansch, P. G. Sammens, J. B. taylor, and C. A. Ramsden, Eds. P. 295, Pergamon Press, 1990, incorporated herein by reference). ClogP values may be calculated by using the “CLOGP” program available from Daylight Chemical Information Systems Inc. of Irvine, Calif. U.S.A.

(5) Boiling Point
Boiling point is measured by ASTM method D2887-04a, “Standard Test Method for Boiling Range Distribution of Petroleum Fractions by Gas Chromatography,” ASTM International.

(6) Delivery Index Calculation
The Delivery Index for a particle is calculated using the following equation:

$$Delivery\_Index = \frac{[ {( \frac{μ}{σ} )}_{Particle\_Size} {( \frac{{f}_{0}}{f} )}_{Fracture\_Stress} ( \frac{L / {L}_{0}}{t / μ} ) ]}{100}$$

### Where

μ is the average particle diameter
σ is the standard deviation of the average particle diameter
f₀ is the minimum in-use fracture strength required to break the microcapsule
f is the measured Fracture Strength
(L/L₀)/(t/μ) is the Active Retention Ratio
t is the shell thickness of the particle
   The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the DETAILED DESCRIPTION OF THE INVENTION are, in the relevant part, incorporated herein by reference; the citation of any document is not to be construed as an admission that it is prior art with respect to the present invention. To the extent that any meaning or definition of a term or in this written document conflicts with any meaning or definition in a document incorporated by reference, the meaning or definition assigned to the term in this written document shall govern.

Except as otherwise noted, the articles “a,” “an,” and “the” mean “one or more.”

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A structured liquid composition comprising:
a. a liquid matrix comprising:
i. a fibrous network structurant at a level of from about 0.05% to about 1% by weight of said composition, said fibrous network structurant comprising:
1. a non-polymeric crystalline hydroxyl-functional materials;
2. a bacterial cellulose network; and
3. mixtures thereof;
ii. water at a level of from about 0.1 to about 75% by weight of said composition; and
b. a plurality of encapsulated actives at a level of from about 0.5% to about 70% by weight of said composition.

2. The composition of claim 1, wherein said level of water is greater than about 1% to about 30%, and said level of said plurality of encapsulated actives is greater than about 10% to about 70%.

3. The composition of claim 2, wherein said encapsulated active consists essentially of a perfume.

4. The composition of claim 1, further comprising an emulsifier and a formaldehyde scavenger.

5. The composition of claim 1, furthering comprising from about 0% to about 4% of a salt.

6. The composition of claim 1, wherein said composition is essentially free of a salt.

7. The composition of claim 1, further comprising an additional structuring agent at a level of from about 0.01% to about 5% by weight of said composition.

8. The composition of claim 1, wherein said composition is essentially free of any additional structuring agent.

9. The composition of claim 1, wherein said fibrous network structurant comprises a fiber having a length to diameter aspect ratio of from about 10 to about 100,000.

10. The composition of claim 1, wherein said encapsulated active comprises a friable microcapsule, a moisture-activated microcapsule, a heat-activated microcapsule, or combinations thereof.

11. The composition of claim 1, wherein said encapsulated material comprises an encapsulated active selected from the group consisting of: flavors, perfumes, softening agents, anti-static agents, crisping agents, water/stain repellents, stain release agents, refreshing agents, anti-microbial agents, disinfecting agents, wrinkle resistant agents, wrinkle release agents, odor resistance agents, malodor control agents, abrasion resistance and protection agents, solvents, insect/pet repellents, wetting agents, UV protection agents, skin/fabric conditioning agents, skin/fabric nurturing agents, color protection agents, dye fixatives, dye transfer inhibiting agents, silicones, preservatives and anti-microbials, fabric shrinkage-reducing agents, perfume microcapsules, brighteners, hueing dyes, bleaches, chelants, antifoams, anti-scum agents, whitening agents, and combinations thereof.

12. The composition of claim 1, wherein said encapsulated material and said fibrous network structurant comprise a zeta potential differential of from about 0.1 to about 2.

13. The composition of claim 1, further comprising a pH of from about 3 to about 10.

14. A process of making a structured liquid composition comprising an encapsulated active comprising:
a. supplying a fibrous network structurant;
b. supplying a encapsulated active slurry; and
c. combining said fibrous network structurant with said encapsulated active slurry to form a mixture;
d. subjecting said mixture to a pressure drop below 100 psi across a mixing chamber to form said composition.

15. The process of claim 14, further comprising a step for forming said fibrous network structurant by combining a non-polymeric crystalline hydroxyl-functional materials, a bacterial cellulose network, or a combination thereof with an aqueous solution and a power density of from about 1 W/Kg to about 10̂9 W/kg of said premix.

16. The process of claim 14, wherein said process is a batch process comprising a step of from about 1 W/Kg to about 10̂9 W/kg of said composition.

17. The process of claim 14, wherein said step of supplying said fibrous network structurant premix comprises:
a. combining a fibrous network structurant with water; and
b. activating said fibrous network structurant with said water to form said fibrous network structurant premix.

18. The process of claim 14, further comprising a step of maintaining a salt concentration of less than about 4%.

19. The process of claim 14, wherein said step of subjecting said mixture to a pressure drop comprises a pressure drop below 4 psi; and wherein said step of subjecting said mixture to a pressure drop is performed over a time period of 2 seconds.

20. A method of making a structured composition comprising:
a. allowing a user to select a type of composition;
b. supplying a fibrous network structurant;
c. supplying an encapsulated active slurry based on the selection made in step a); and
d. combining said fibrous network structurant with said encapsulated active slurry to form a structured composition.
